(19) United States
(12) Patent Application Publication
(10) Pub. No.: US 2016/0058350 A1
(43) Pub. Date: Mar. 3, 2016

(54) SYSTEM AND METHOD FOR CONTROLLING ONE OR BOTH OF SENSOR FUNCTIONALITY AND DATA ACCESS BASED ON BIOMETRICS DATA

(71) Applicant: COVIDIEN LP, Mansfield, MA (US)

(72) Inventors: Casey V. Medina, Westminster, CO (US); Charles Haisley, Boulder, CO (US); Michelle L. Hodge, Longmont, CO (US)

(21) Appl. No.: 14/939,872

(22) Filed: Nov. 12, 2015

Related U.S. Application Data
(63) Continuation of application No. 12/494,986, filed on Jun. 30, 2009, now abandoned.

(51) Int. Cl. A61B 5/1455; A61B 5/117

(52) U.S. Cl. CPC A61B 5/14552; A61B 5/1172; A61B 5/1176; A61B 2560/0475

(57) ABSTRACT

The present disclosure is generally directed to maintaining sensor quality standards and/or preventing improper remanufacture of sensors, such as pulse oximetry sensors. Present embodiments may include a system that includes a biometric measurement device configured to obtain biometric data of a patient and a pulse oximetry sensor configured to obtain physiological data from a patient. The pulse oximetry sensor may include a memory device configured to receive and to store biometric data obtained during an initial reading of the patient by the biometric measurement device. The system may include a processor configured to obtain subsequent biometric data from the biometric device, to compare the subsequent biometric data to the biometric data obtained during the initial reading, and to prevent the pulse oximetry sensor from obtaining physiological data if the subsequent biometric data does not correspond to the biometric data obtained during the initial reading.

### CROSS-REFERENCE TO RELATED APPLICATIONS

This application is a continuation of U.S. patent application Ser. No. 12/494,986, entitled “System and Method for Controlling One or Both of Sensor Functionality and Data Access Based on Biometrics Data,” filed Jun. 30, 2009, the disclosure of which is hereby incorporated by reference in its entirety for all purposes.

The present disclosure is related to U.S. Pat. No. 9,010,634, entitled “System and Method for Linking Patient Data to a Patient and Providing Sensor Quality Assurance,” and to U.S. Pat. No. 8,505,821, entitled “System and Method for Providing Sensor Quality Assurance,” each of which is herein incorporated by reference in its entirety for all purposes. Both U.S. Pat. No. 9,010,634 and U.S. Pat. No. 8,505,821 were filed concurrently with the parent of the present application and include the same inventors as the present application.

### BACKGROUND

The present disclosure relates generally to physiological monitoring instruments and, in particular, to a sensor that utilizes a physiological biometric characteristic of a patient to control access to patient data by linking the patient data with a patient and/or to guard against sensor modification and misuse by linking the patient to the sensor.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

There are numerous techniques and systems for monitoring a patient's physiology. For example, pulse oximetry may be used to continuously monitor physiologic characteristics of a patient. Pulse oximetry may generally be defined as a non-invasive technique that facilitates monitoring of a patient's blood characteristics. For example, pulse oximetry may be used to measure blood oxygen saturation of hemoglobin in a patient's arterial blood and/or the patient's heart rate. Specifically, in pulse oximetry, blood characteristic measurements may be acquired using a non-invasive sensor that passes light through a portion of a patient's blood perfused tissue and that photoelectrically senses the absorption and scattering of light through the blood perfused tissue. Various wavelengths of light may be used that may or may not pass through certain blood constituents. Indeed, a typical pulse oximetry sensor includes at least two light emitters that emit different wavelengths of light, and a light detector. Based on how much light at certain wavelengths is emitted and detected, and based on absorption and scattering characteristics of certain blood constituents, an estimate of the blood content may be made based on the detection results. For example, a typical signal resulting from the sensed light may be referred to as a plethysmographic waveform, which is a measurement of the absorbed and scattered light at different wavelengths.

Once acquired, the plethysmographic waveform may be used with various algorithms to estimate an amount of blood constituent in the tissue, as well as other physiologic characteristics. This and other types of data may be collected over time to provide trend data or historical data for a patient. This trend data or historical data may be stored for use in assessing a patient's condition, reviewing a patient's progress, or the like. However, it is now recognized that such stored data can potentially be disassociated with a patient. For example, the data may be stored on a device or system that is used to monitor multiple patients, and the data for a particular patient may be confused with that of a different patient having been monitored or otherwise addressed by the device or system. Accordingly, it is now recognized that a technique for creating a strong association between such data and the appropriate patient may be desirable.

Some conventional sensors, such as conventional pulse oximetry sensors, may include an information element that stores information that can be read by a monitoring device to facilitate proper use of the sensor. For example, a pulse oximeter sensor may include a memory or a resistor that can be read by an oximeter. The information stored on the information element may include parameters about the sensor. For example, with regard to a pulse oximeter sensor, the information may indicate sensor type (e.g., neonatal, pediatric, or adult), the wavelengths of light produced by the emitters, and so forth. Certain data stored in the pulse oximeter sensor, such as the wavelengths of light associated with the emitters of the sensor, may be important for proper blood characteristic measurement. This information may be utilized in algorithms for determining values for one or more measured blood characteristics. Further, the information element may be utilized for security and quality control purposes. For example, the information element may ensure proper operation by preventing the sensor from functioning with improperly configured or unauthorized devices.

Due to the function of the information element, it is often necessary for sensor operation. Accordingly, the information element is often included in unapproved remanufactured sensors to enable their operation. However, such unauthorized remanufactured sensors may be unreliable and fail to function properly. Indeed, improper remanufacturing of a sensor or tampering with the sensor can impact the quality and reliability of the sensor, especially when a sensor includes an information element with pertinent operational data stored thereon. For example, improper remanufacturing of a sensor may result in consistently incorrect measurements and/or cause malfunctions by coupling incompatible sensor components together. In a specific example, an information element for a neonatal oximeter sensor may be improperly incorporated into the body of an adult oximeter sensor during remanufacture. Thus, the information element incorporated into the remanufactured sensor may include settings for a neonatal application that do not correspond to the wavelengths associated with the light emitters of the remanufactured sensor, which correspond to an adult application. Such remanufacturing can cause improper operation and incorrect measurement of physiological characteristics.

### BRIEF DESCRIPTION OF THE DRAWINGS

Advantages of present embodiments may become apparent upon reading the following detailed description and upon reference to the drawings in which:

FIG. 1 is a perspective view of a patient physiological data measurement system in accordance with an exemplary embodiment of the present disclosure;

FIG. 2 is a perspective view of a patient physiological data measurement system including an attached biometric measurement device in accordance with an exemplary embodiment of the present disclosure;

FIG. 3 illustrates a front view and a back view of a sensor including a processor and a memory configured to store unique biometric data for a patient for use is confirming authenticity in accordance with an exemplary embodiment of the present disclosure; and

FIG. 4 illustrates a flow diagram of a process in accordance with an exemplary embodiment of the present disclosure.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present disclosure relate, in general, to a sensor for measuring patient physiological characteristics. For example, present embodiments may include a pulse oximeter sensor that functions to measure oxygen content in a patient's blood. More particularly, present embodiments are directed to sensor designs and corresponding hardware and/or software that facilitate associating patient data with a particular patient and preventing remanufacture of the sensor or making such remanufacture impractical. Indeed, it is now recognized that a strong association of patient data with the appropriate patient is desirable. Further, it is now recognized that unauthorized remanufacture of a sensor can create issues with proper operation of the sensor, and, thus, it is desirable to guard against such practices. Accordingly, present embodiments are directed to improving sensor quality assurance by preventing sensor remanufacture.

Specifically, embodiments of the present disclosure relate to a system including a sensor configured to store biometric data that uniquely identifies a patient. Unlike mere physiologic data, unique biometric data includes biometric data with a quality that distinguishes individuals from one another. For example, a measurement of a patient's fingerprint may be representative of unique biometric data because it can readily be used to distinguish one patient from another. The sensor may include a memory that receives such biometric data from a biometric sampling device. The biometric sampling device may include one or more of a fingerprint scanner, a retina scanner, an iris scanner, a voice recognition device, a facial feature recognition device, a DNA testing device, or any of various other devices that are capable of measuring a biometric parameter unique to a patient. The biometric sampling device may be a component of a monitor (e.g., a pulse oximeter) or a component of the sensor itself.

The sensor and/or the monitor may be configured to utilize the unique biometric data to associate other patient data, such as historical pulse oximetry data, with the patient that provided the biometric data. Additionally, the functionality of the sensor may be associated with the biometric data. Thus, in accordance with present embodiments, access to the historical data and/or operation of the sensor may be controlled by comparing the stored biometric data with data initially scanned into a memory device (e.g., into a sensor memory) by the biometric sampling device. For example, present embodiments may periodically (e.g., when a sensor is activated) require that a biometric characteristic of a patient be measured and matched with biometric data stored on the sensor to enable operation of the sensor or allow access to patient data. This may protect the confidentiality of patient data and/or avoid association of a patient's data with a different patient. Further, this may substantially prevent remanufacture of the sensor by, for example, preventing operation of the sensor without a correspondence between the current patient's biometric measurements and data stored on the sensor.

In accordance with present embodiments, patient-specific data (e.g., physiologic trend data acquired over time for a particular patient) may be stored in a sensor memory, a monitor memory, or in a central storage system, such as a computer network of a hospital. It may be desirable to store such data within the sensor to facilitate the ability to control access to the information by controlling access to the sensor, which may be achieved by controlling whether the sensor will function at all. Additionally, storing the information on the sensor may facilitate access to the information and association of the information with the patient when the patient is moved around. Indeed, when such data is stored in the sensor itself, it may be desirable to keep the sensor attached to the patient as the patient is moved around the hospital. This may allow the patient's historical data to go with the patient to different monitors in different areas of the hospital. Similarly, having such data available on a central network may facilitate access to a patient's data as the patient is moved around.

Present embodiments provide a system and technique for confirmation of a correspondence between stored patient data and the patient, and/or confirmation of the authenticity of a sensor assigned to the patient. For example, the patient, the sensor, and/or information obtained by the sensor may be linked by acquiring one or more measurements of unique biometric characteristics of the patient and storing the related data in the sensor. Subsequently, direct biometric measurements may be compared with the stored biometric data to confirm correspondence and/or authenticity. In some embodiments, the data acquired via the sensor may be linked with the patient by, for example, encoding the data acquired by the sensor (e.g., pulse oximetry data) based on data representative of a patient's unique biometric characteristic. For example, numeric data representative of a fingerprint scan and obtained by scanning the patient's finger with a fingerprint scanner may be stored on a sensor and utilized to encode subsequently acquired physiologic measurements that are obtained and/or stored on the sensor. Thus, subsequent access to the encoded physiologic measurements may require a subsequent scan of the patients fingerprint to decode the physiologic measurements for presentation. Such a technique may be utilized to confirm that a single patient is using the sensor.

The patient's unique biometric characteristic may be utilized to identify the patient and to confirm that the sensor and/or historical data correspond to the proper patient. For example, in order to access a particular patient's historical data, it may be necessary to scan the patient's fingerprint or iris with an optical scanner or test the patient's DNA. Unless the patient has the biometric characteristic that has been linked to the sensor and/or the patient-specific historical data, it will not be accessible. This may avoid issues relating to accessing inaccurate information for a particular patient. Indeed, certain biological features of a patient may be scanned or tested to verify that the sensor attached to the patient was assigned to the patient, and, thus, confirm the data stored therein is correlated to the patient. Likewise, the patient's unique biometric data may be obtained to verify correspondence to data stored on a central system that has been encoded based on the biometric data or otherwise associated with the biometric data. Additionally, the link between the patient's unique biometric data and the sensor may also present a substantial obstacle to improper remanufacturing of the sensor because, as will be discussed in further detail below, unless the proper biometric data is periodically updated from the patient via a measurement (e.g., a retina scan), the sensor may not function.

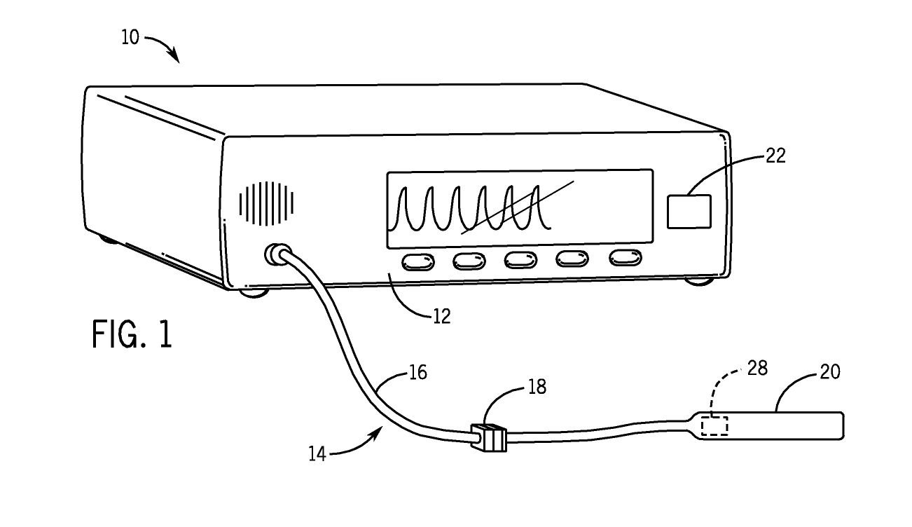

FIG. 1 is a perspective view of a patient physiological data measurement system in accordance with an exemplary embodiment of the present disclosure. Specifically, FIG. 1 includes a pulse oximeter system, which is generally indicated by reference numeral 10. The system 10 includes a specially programmed computer or monitor 12 (e.g., a pulse oximeter) that communicatively couples via a cable or wirelessly to a sensor 14. The sensor 14 includes a sensor cable 16, a connector plug 18, and a body 20 configured to attach to a patient. In the illustrated embodiment, the body 20 of the sensor 14 is generally planar and configured for application to a patient's skin.

In accordance with present embodiments, the body 20 of the sensor 14 may be configured to couple with a patient's earlobe, finger, foot, forehead, or other locations on the patient that facilitate non-invasive measurement of desired physiological data (e.g., pulse rate and/or blood oxygen saturation). In another embodiment, the sensor 14 may be configured for invasive operation and the body 20 may be configured for insertion into a patient. The sensor cable 16 and connector plug 18 may enable electronic communication from the sensor 14 to the monitor 12, and facilitate coupling and/or decoupling the sensor 14 from the monitor 12. In some embodiments, the sensor 14 may couple directly to the monitor 12 via the sensor cable 16. In other embodiments, the sensor 14 may communicate with the monitor 12 wirelessly (e.g., via radio waves) and may not include the cable 16 or the connector plug 18.

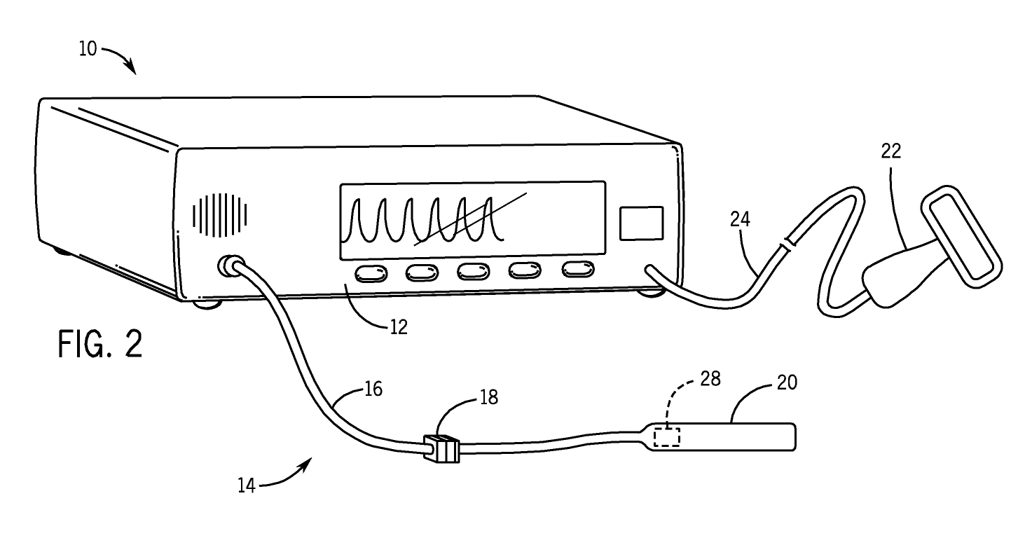

The sensor 14 may cooperate with a biometric measurement device (BMD) 22 that may be integral with or separate from the sensor 14. The BMD 22 may include one or more of various devices capable of measuring biometric characteristics of a patient that facilitate identification of the patient, such as a fingerprint scanner, a retina scanner, an iris scanner, a voice recognition device, a facial feature recognition device, a palm geometry scanner, a DNA testing device, or the like. In some embodiments, the BMD 22 may be integral with a main body of the monitor 12, as illustrated in FIG. 1. Specifically, the BMD 22 illustrated in FIG. 1 includes an optical scanner that may be capable of scanning a patient's fingerprint or palm. In such embodiments, to obtain the unique biometric data via the BMD 22, the physical feature of the patient being scanned (e.g., the patient's finger) may be passed in front of the BMD 22. Thus, the BMD 22 may scan the physical feature and convert it into data. This biometric data can be communicated to a memory of the monitor 12 or the sensor 14 and so forth. However, in some embodiments, as illustrated in FIG. 2, the BMD 22 (e.g., a scanning gun or a DNA sampling device) may be coupled to the monitor 12 via a biometric device cable 24. The cable 24 may be sufficiently long to facilitate obtaining biometric data in various locations relative to the monitor 12. For example, the BMD 22 may be configured to measure certain physiological features that are not easily placed in front of the monitor 12, such as facial features. Thus, it may be desirable for the cable 24 to be long enough to facilitate movement of the BMD 22 over the patient (e.g., the patient's face) instead of having to move the patient in front of the monitor 12. In fact, in some embodiments, the BMD 22 may wirelessly communicate with the monitor 12 to facilitate obtaining biometric data without moving the patient into close proximity with the monitor 12.

Additionally, while the embodiment illustrated in FIG. 2 shows the BMD 22 coupled directly to the monitor 12, in other embodiments, the BMD 22 may be coupled to the monitor 12 via the sensor cable 16. Furthermore, in some embodiments, the BMD 22 may not communicate with the monitor 12, but, rather, the BMD 22 may communicate directly with the sensor 14 or an information element 28 (e.g., a sensor memory). This may be facilitated by more closely incorporating the BMD 22 with the sensor 14. In fact, a small scanning or testing mechanism, such as a small fingerprint reader or the like, may be incorporated into the sensor 14 to operate as the BMD 22 in accordance with present embodiments.

With regard to linking patient-specific data to a patient, present embodiments may be capable of reading or otherwise obtaining unique biometric data for a patient with the BMD 22 and utilizing a processor and a memory to electronically associate the biometric data with data stored within and/or acquired by the sensor 14. For example, present embodiments may include an iris scanner as the BMD 22 and a sensor memory as the information element 28. The BMD 22 may be configured to obtain unique biometric data by scanning a patient's iris, and configured to store the unique biometric data on the sensor memory. Thus, the patient and the sensor may be linked by the stored biometric data on the sensor memory. The sensor memory may also store historical patient data acquired by the sensor 14, such as historical pulse oximetry data. Thus, the historical patient data and the unique biometric data may be associated by common storage. In accordance with present embodiments, storing the unique biometric data on the same memory with the historical patient data may include electronically linking the unique biometric data with the patient data. For example, the patient data may be encoded based on all or certain components of the biometric data such that it cannot be accessed without periodically retrieving the biometric data via the BMD 22 when certain predefined conditions are present, such as each time the sensor 14 is activated. Accordingly, the sensor 14 and the stored data may be specifically linked to the patient based on the patient's unique biometrics. In other embodiments, other memory devices may be employed in addition to or instead of the sensor memory. For example, a central system memory or a pulse oximeter memory may be utilized to store patient specific data that is associated with the unique biometric data (e.g., encoded based on the biometric data). Subsequent access to the centrally stored data may require entry of the unique biometric data via the BMD 22. Regardless of the location of the memory, such subsequently acquired unique biometric data may be required to match the initially scanned data to access the historical data or to even function with memory and/or the sensor 14.

Also, as indicated above, unique biometric data acquired via the BMD 22 and the information element 28 within the sensor 14 may cooperate to prevent efficient remanufacture of the sensor 14. For example, in one embodiment, the information element 28 includes a sensor memory that operates to prevent the sensor 14 from functioning to acquire or supply data unless biometric data matching that stored in the memory is communicated via the BMD 22. Such data acquisition by the BMD 22 may be required periodically based on circumstances or based on timing. For example, the sensor may function after biometric data is confirmed until the sensor is disabled (e.g., removed from a monitor) and then require another confirmation when the sensor is activated again (e.g., reconnected to a monitor). Thus, unless the unique biometric data for a particular patient remains available, which generally requires the presence of the patient, the sensor 14 will not function. Accordingly, the sensor 14 essentially limits itself to use with a single patient, and subsequent use of the sensor 14 with a different patient that was not initially linked to the sensor 14 will fail because the biometric data for both patients will not match. Indeed, the biometric data from the linked patient, which is stored in memory, will not match the biometric data that can be acquired from the patient subsequently attempting to use the sensor 14.

The link with the patient provided via the stored biometric data may also prevent remanufacture of the sensor 14 because the critical features for operation of the sensor 14 will require confirmation of the stored biometric data. Without having the linked patient present, counterfeit biometric data would be required to get access to the data and/or the sensor, which would be difficult to obtain and efficiently distribute. Further, if a potential remanufacturer attempted to simply remove the stored biometric data that links the historical data and/or sensor to the patient, present embodiments may completely disable the sensor. For example, a sensor memory in accordance with present embodiments may store encryption data along with the biometric data. Thus, removal of the biometric data may also require removal of encryption data that is required for operation with a monitor.

As a specific example of an operational procedure in accordance with present embodiments, when the sensor 14 is connected to the monitor 12, the monitor 12 may recognize the connection to the sensor 14 and a user may be prompted to obtain unique biometric data with the BMD 22. For example, the monitor 12 may prompt a user via a video or audio message to scan a patient's fingerprint. Further, a memory field in a memory device of the sensor 14 may be read because the memory field is intended to store the unique biometric data obtained via the BMD 22. If no biometric data is present in the memory field, data retrieved from the BMD 22 may be written to the memory field. Indeed, the data retrieved from the BMD 22 may be transmitted from the monitor to the sensor memory or transmitted directly from the BMD 22 to the sensor memory for storage in the memory field. The patient's unique biometric data may be used to tag or identify data acquired by the sensor 14 as belonging to that particular patient. When the unique biometric data is initially stored in the memory field, the sensor 14 may begin to function, or a second measurement of the patient's unique biometric data may be required to confirm a correspondence between the biometric data stored in the sensor 14 and the biometric data of the patient. This may be initially required or only when certain conditions are present. For example, a patient's retina may be scanned to provide the initial biometric data to enable the sensor 14 to begin functioning, and a second retina scan of the patient may be automatically required or required after the sensor has been deactivated to confirm correspondence and initiate use of the sensor 14 with the monitor 12. Alternatively, if unique biometric data is already present in the memory field when the sensor 14 is initially activated or connected to the monitor 12, the stored data may be compared to unique biometric data obtained via the BMD 22 from the patient on which the sensor 14 is to be used. If the data stored in the sensor memory corresponds to that obtained from the patient, the sensor may be allowed to operate. If it is different, the monitor 12 may reject the sensor 14 and/or indicate that the sensor 14 is not an approved sensor. In some embodiments, the sensor 14 itself may disable its ability to function unless the stored biometric data matches the newly acquired biometric data.

In one embodiment, the initial storage of unique biometric data from the patient may include integrating the unique biometric data with data that is required for sensor operation. For example, the unique biometric data initially stored in the sensor memory may be incorporated with data already stored in the sensor memory such as calibration data, data required to decrypt information provided by the sensor, and so forth. Thus, the biometric data matching the initially acquired biometric data is required for proper sensor operation and/or access to historical data, and acquiring confirmation biometric data from a different patient would prevent further use of the sensor and/or access to the data because unique biometric data is very rarely identical between two different individuals. Further, bypassing this feature with fake biometric data would be difficult. For example, obtaining and reproducing counterfeit biological representations (e.g., a printed representation of a fingerprint) would be exceedingly difficult and inefficient. Indeed, assuming the sensor 14 has already been used and biometric data from a particular patient's fingerprint has already been incorporated into the sensor memory, such a remanufacturing process would require that the patient's fingerprint be duplicated.

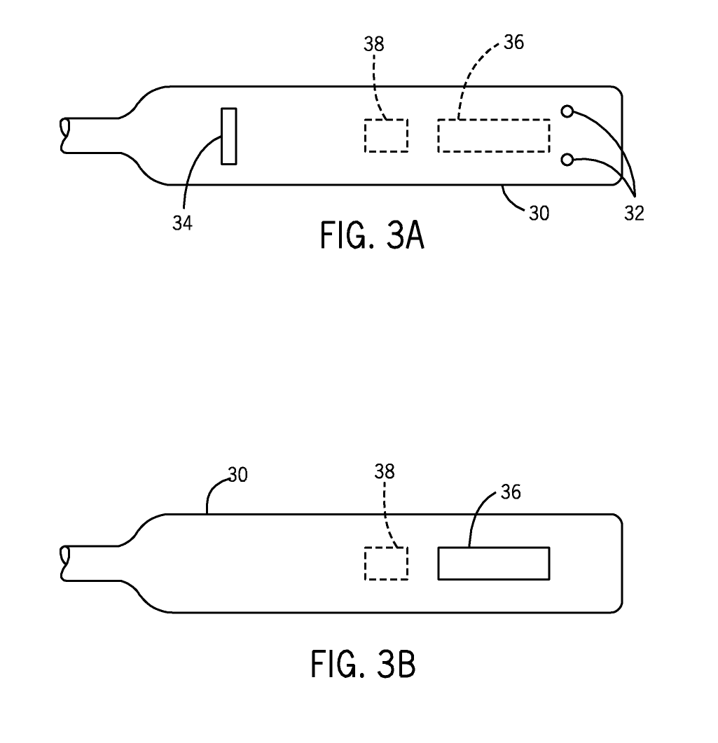

FIGS. 3A and 3B illustrate a sensor 30 in accordance with present embodiments. Specifically, FIG. 3A illustrates a front side of the sensor 30 and FIG. 3B illustrates a back side of the sensor 30. In the illustrated embodiment of FIG. 3, the sensor 30 is a pulse oximeter sensor that includes a pair of emitters 32 (e.g., a red emitter and an infrared emitter) configured to emit light waves, and a photodiode detector 34 that is arranged to detect the emitted light waves. Such sensors are typically configured to attach to a patient's finger, foot, forehead, or earlobe to facilitate measurement of blood characteristics in the associated tissue. For example, the sensor 30 may be adapted to project light from the emitters 32 through the outer tissue of a finger and into the blood vessels and capillaries inside, and detect the emitted light at the detector 34 as the light emerges from the outer tissue of the finger. In operation, the detector 34 may generate a signal based on the detected light and provide the signal to the monitor 12, which may determine blood oxygen saturation based on the signal. For example, the monitor 12 may utilize the signal to display a plethysmographic waveform. Data such as this may be stored in a sensor memory 36, a central system memory, or the like. Further, such data may be linked to a patient by requiring that a certain biological characteristic of the patient be read by the BMD 22 prior to granting access to the stored data. Indeed, the patient data may even be encoded based on the patient's unique biometric data and the monitor 12 and/or sensor 30 may be programmed to require periodic confirmations of the patient's identity via the BMD 22 such that the presence of the patience is required to decipher the patient data.

Further, as discussed above, the sensor 30 may include the sensor memory 36 and a processor 38, which may be internal or external to the sensor 30, and which is configured to store data. For example, the sensor memory 36 may store historical patient data, trend data, and/or data that relates to disabling or enabling the functionality of the sensor 30. In one embodiment, the sensor memory 36 may include a memory device (e.g., ROM) that stores the patient's unique biometric data. A correlation between the biometric data acquired via the BMD 22 and the data stored on the sensor memory 36 may be required for the sensor 30 to function. For example, the processor 38 or a processor in the monitor 12 may compare the data stored in the sensor memory 36 with the data identified by testing a patient's DNA or scanning the patient's fingerprint, retina, iris, or the like with the BMD 22 and determine whether the data matches. If the data does not match, the sensor 30 and/or the monitor 12 may prevent operation of the sensor 30 and/or provide an error indication (e.g., an alarm).

It should be noted that the sensor memory 36 may be manufactured with a blank portion specifically for storing the patient's biometric data. Thus, the sensor 30 may essentially be generic until a particular patient's biometric data is communicated and stored in the sensor memory 36. For example, in use, a patient's fingerprint may be scanned by a fingerprint scanner, and the associated data may be stored in the memory 36 and the sensor 30 may be enabled to operate. Additionally, any data acquired by the sensor 30 may be designated as being associated with the patient's fingerprint data to facilitate association of the data with the patient and/or prevent access to the data without confirming the identify of the patient by scanning the patient's fingerprint. In some embodiments, scanning any of various types of unique biometric data may enable operation. For example, any of a fingerprint, iris, or retina may be scanned and recognized as data for later use in confirmation of patient identity and the like. Once a proper type of data has been scanned into the memory 36, the sensor 30 may be enabled to operate. However, subsequent operation (e.g., operation after the sensor 30 has been powered down or detached from the monitor 12) may be prevented unless an identical unique biometric value is scanned and communicated to the memory 36. Accordingly, the sensor 30 may be originally manufactured such that it can be electronically coupled with any unique patient biometric by storing that data on the sensor 30. However, reuse of the sensor 30 is prevented unless unique biometric data identical to that initially scanned into the memory 36 is measured and utilized to activate the sensor 30 for subsequent uses.

Various techniques may be utilized to prevent copying and/or erasing the sensor memory 36 for reuse. For example, to prevent copying of data from the sensor memory 36, the data corresponding to the initially acquired biometric measurements may be scattered in different memory locations. That is, when the sensor 30 is first assigned to the patient and the patient's biometric data is first stored in the sensor memory 36 as reference data for use in later confirming patient identity, it may be broken up and stored in various locations. Thus, a distinction between the reference data and other data (e.g., historical data, encryption data) in the sensor memory 36 may be difficult to discern. Further, encryption of the data may be employed. In one embodiment, an encryption feature, such as an encrypted signature, may be included on the sensor memory 36. Accordingly, if the sensor memory 36 is erased in an attempt to remanufacture the sensor 30, the encryption feature will also be erased, which will prevent further use of the sensor 30 by monitors that require such an encryption feature.

The biometric data initially stored by the sensor 39 may be utilized by a hospital network to identify the patient. For example, upon checking into a hospital, certain procedures may be preformed on the patient to acquire one or more unique biometric values for the patient, the data for these values may be encrypted and linked to the patient. In other words, within the hospital network the patient and the patient's records may be electronically associated the unique biometric data acquired from the patient. Thus, each time the patient's unique biometric data is read, the association with the patient may be recognized.

Remanufacture of a sensor that was originally assembled in accordance with present embodiments may be achieved, but may be difficult. For example, a used sensor may be sterilized and reassembled to include a copy of a patient's biometric data, such a copy of a patient's fingerprint on a piece of paper that is capable of being scanned by the BMD 22, or some other manner of data that is capable of bypassing the BMD 22. Thus, the copied biometric data may be utilized to satisfy the requirement of having identical data without the presence of the initial patient. Further, various components may be replaced and/or reused. For example, reprocessing may include erasing only those features of the memory that store the initial unique biometric data that is utilized for later comparison with the unique biometric data acquired via the BMD 22 to confirm authenticity. However, this would require knowledge of the location of such data and the ability to erase it without erasing other data. Further, reprocessing may include erasing all of the information on the information element (e.g., memory) of a sensor, which would also erase encrypted information required for operation. Once the information element is blank, new encryption information could be included with a blank biometric data code field (e.g., a field for storing initial unique biometric data for later comparison with unique biometric data acquired via the BMD 22) by an entity with the proper encryption knowledge. In some embodiments, this may include wiping a memory and pointing to a new memory section for storing encryption data and/or unique biometric data.

It should be noted that the information element may include any of various different types of memory in accordance with present embodiments. For example, write-once memory (WOM) and/or rewritable memory (EPROM, EEPROM) may be utilized. These different types of memory would have an impact on attempts to remanufacture a sensor. With regard to write-once memory, for example, the write-once memory would be invalidated by any attempt to erase the biometric data code field because it cannot be rewritten. With regard to rewritable memory or write-many memory, the biometric data code field could be erased and rewritten. However, such a memory may be prevented from functioning in accordance with present embodiments because of encryption of the biometric data field, scattering of the biometric data field, and/or a special field indicating that the biometric data has been written (e.g., a recorded date of rewriting).

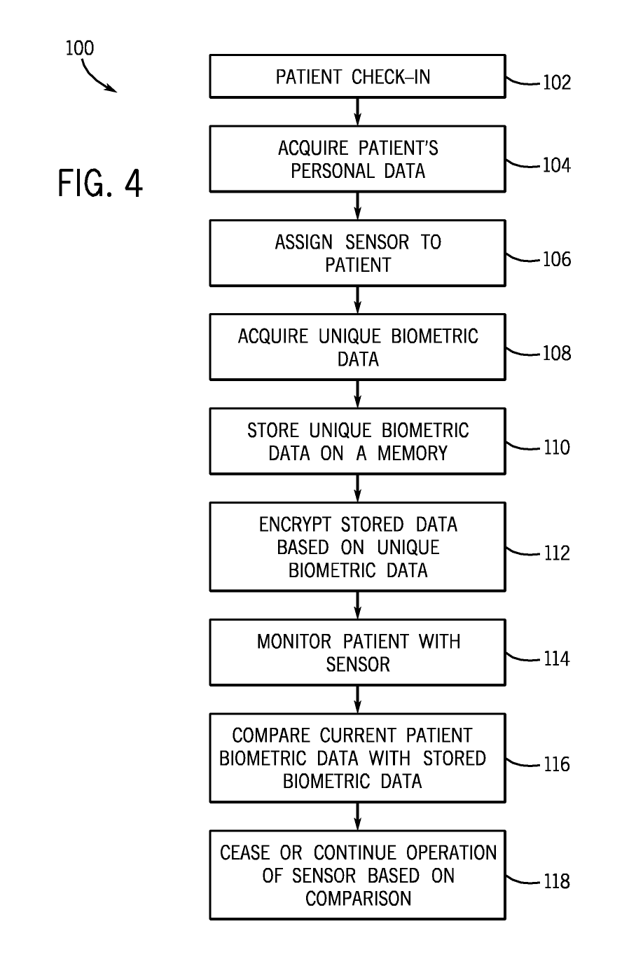

FIG. 4 illustrates a flow diagram of a process in accordance with an exemplary embodiment of the present disclosure. The process is generally indicated by reference numeral 100. The process 100 may represent various procedures that may be employed in accordance with present embodiments or using features in accordance with present embodiments. Specifically, the process 100 includes a procedure for admitting and monitoring a patient using a sensor that has been linked to the patient via the patient's unique biometric data. The process 100 also includes preventing multiple uses of the sensor with different patients based on detection and storage of the original patient's unique biometric data.

In the illustrated embodiment, the process 100 begins, as illustrated by block 102, with a patient checking in to a hospital, a clinic, a procedural area, or the like. For example, block 102 may represent a patient checking in for surgery or for a sleep test. During or after check-in, personal information for the patient may be acquired, as represented by block 104. This may include inputting the patient's address, social security number, insurance, and so forth. In some embodiments, the patient's personal information may already be available for retrieval from a central system. Accordingly, block 104 may simply represent downloading the patient's stored personal data or merely confirming that the patient is already in the system.

Once the patient has been checked in, the patient may be assigned a sensor, as represented by block 106. In some embodiments, the sensor may be assigned to the patient during or before the patient has checked in. In one embodiment, for example, upon signing the patient in for a procedure, a pulse oximeter sensor may be attached to the patient. Block 106 may also represent storing the patient's personal data on a memory of the sensor and/or indicating that the specific sensor has been assigned to the patient on a central storage system based on a unique sensor identifier (e.g., a serial number).

Once the patient has been assigned a sensor, the sensor and the patient may be linked by the patient's unique biometric data. This may include measuring (e.g., scanning) one or more unique biological features of the patient to acquire the unique biometric data, as represented by block 108, and then storing the unique biometric data on a memory (e.g., a sensor memory), as represented by block 110. For example, block 108 may include acquiring the biometric data with one or more biometric sampling devices such as a fingerprint scanner, a retina scanner, an iris scanner, a voice recognition device, a facial feature recognition device, a DNA testing device, or any of various other devices that are capable of measuring a biometric parameter unique to a patient. It should be noted that the biometric sampling device may be integral with the sensor or separate (e.g., a component of a monitor).

In addition to storing this unique biometric data, as represented by block 110, present embodiments may include linking the sensor to the patient by encoding or encrypting all or a portion of the data relating to the sensor (e.g., measurements acquired by the sensor, calibration information, and patient information) based on the stored biometric data, as represented by block 112. For example, pulse oximetry measurements acquired by the sensor may be encoded such that they are indecipherable without confirmation that the current patient's unique biometric data matches the unique biometric data originally stored in the memory. For example, subsequent scanning of the patient's fingerprint may be required to substantially match that stored in memory to enable decoding of stored historical pulse oximetry data.

In operation, the sensor may be utilized to monitor the patient, as illustrated by block 114. In other words, the sensor may be utilized to acquire physiologic data, such as pulse oximetry data, from the patient and store the historical physiologic data in a memory (e.g., a sensor memory or monitor memory). In some embodiments, this physiologic data may be encrypted based on the unique biometric data acquired from the patient with the biometric sampling device, which may be incorporated in the sensor but separate from the actual sensing features that acquire the physiologic data.

Periodically, during operation or based on breaks in operation (e.g., when the sensor is moved from one monitor to another, powered down, or removed and then reattached to the same monitor), the sensor and/or monitor may require confirmation that the patient being monitored was assigned the sensor by comparing the patient's unique biometric data with that stored in memory. Accordingly, the biometric sampling device may be required to periodically be utilized to obtain the patient's unique biometric data and then that data may be compared with the previously stored unique biometric data, as illustrated in block 116. For example, when a sensor including stored unique biometric data is attached to a monitor, it may automatically indicate that the patient's fingerprint should be scanned to determine whether the patient's unique biometric data matches the unique biometric data stored in the sensor. In some embodiments, the scanning may automatically be performed by a biometric sampling device (e.g., a small fingerprint scanner) integral with the body of the sensor and arranged to continually have access to the patient's fingerprint.

If the patient's biometric data does not correspond to (e.g., match or substantially match) the previously stored unique biometric data, the sensor may cease to operate completely, only partially function, and/or provide an error message, as illustrated in block 118. For example, if the sensor or some critical component of the sensor (e.g., the sensor memory) has been removed from the originally assigned patient and attached to a different patient, the sensor may be prevented from fully functioning or functioning at all because the patient's biometric data will not correspond to that of the previous patient, and the previous patient's biometric data is stored in the memory. Thus, present embodiments may prevent sensor misuse. For example, the original patient may be an adult and second patient may be a child, while the sensor may be specifically designed for an adult patient. Accordingly, disabling the sensor may prevent erroneous measurements of the second patient's physiologic data.

While the embodiments set forth in the present disclosure may be susceptible to various modifications and alternative forms, specific embodiments have been shown by way of example in the drawings and have been described in detail herein. However, it should be understood that the disclosure is not intended to be limited to the particular forms disclosed. Indeed, the present techniques may not only be applied to measurements of blood oxygen saturation, but these techniques may also be utilized for the measurement and/or analysis of other physiological characteristics. The disclosure is to cover all modifications, equivalents, and alternatives falling within the spirit and scope of the disclosure as defined by the following appended claims.

1. A system, comprising:
a biometric measurement device configured to obtain biometric data of a patient;
a pulse oximetry sensor configured to obtain physiological data from the patient comprising:
a light emitter configured to direct light at the patient;
a light detector configured to receive light from the patient that is indicative of the physiological data; and
a memory device configured to receive and to store biometric data obtained during an initial reading of the patient by the biometric measurement device; and
a processor programmed to obtain subsequent biometric data from the biometric device, to compare the subsequent biometric data to the biometric data obtained during the initial reading, and to prevent the pulse oximetry sensor from obtaining physiological data when the subsequent biometric data does not correspond to the biometric data obtained during the initial reading.

2. The system of claim 1, wherein the pulse oximetry sensor comprises the processor or the biometric measurement device.

3. The system of claim 1, wherein the processor is programmed to enable the pulse oximetry sensor to obtain physiological data when the subsequent biometric data corresponds to the biometric data obtained during the initial reading.

4. The system of claim 1, wherein the processor is programmed to prompt acquisition of the subsequent biometric data via the biometric measurement device periodically during a patient monitoring session.

5. The system of claim 1, wherein the processor is programmed to prompt acquisition of the subsequent biometric data via the biometric measurement device upon connection of the pulse oximetry sensor to a pulse oximetry monitor.

6. The system of claim 1, wherein the processor is programmed to prompt acquisition of the subsequent biometric data via the biometric measurement device upon powering on the pulse oximetry sensor.

7. The system of claim 1, wherein the memory device is configured to store biometric data of only a single patient and the pulse oximetry sensor is configured to be used with only the single patient.

8. The system of claim 1, wherein the processor is programmed to encrypt the physiological data based on the biometric data obtained during the initial reading.

9. A sensor, comprising:
a light emitter configured to direct light at the patient;
a light detector configured to receive light from the patient that is indicative of the physiological data;
a biometric measurement device configured to obtain biometric data of a patient;
a memory device configured to receive and to store biometric data obtained during an initial reading of the patient by the biometric measurement device; and
a processor programmed to obtain subsequent biometric data from the biometric device, to compare the subsequent biometric data to the biometric data obtained during the initial reading, and to enable the pulse oximetry sensor to obtain physiological data when the subsequent biometric data does corresponds to the biometric data obtained during the initial reading.

10. The sensor of claim 9, wherein the processor is programmed to prevent the pulse oximetry sensor from obtaining physiological data when the subsequent biometric data does not correspond to the biometric data obtained during the initial reading.

11. The sensor of claim 9, wherein the processor is programmed to prompt acquisition of the subsequent biometric data via the biometric measurement device periodically during a patient monitoring session.

12. The sensor of claim 9, wherein the processor is programmed to prompt acquisition of the subsequent biometric data via the biometric measurement device upon connection of the pulse oximetry sensor to a pulse oximetry monitor or upon powering on the pulse oximetry sensor.

13. The sensor of claim 9, wherein the memory device is configured to store biometric data of only a single patient and the pulse oximetry sensor is configured to be used with only the single patient.

14. The sensor of claim 9, wherein the processor is programmed to encrypt the physiological data based on the biometric data obtained during the initial reading.

15. The sensor of claim 9, wherein the biometric measurement device comprises an optical reader configured to scan a fingerprint, an iris, a retina, a face, or a palm to obtain biometric data of the patient.

16. The sensor of claim 9, wherein the biometric measurement device comprises a DNA testing device.

17. A method of operating a pulse oximetry sensor, comprising:
acquiring a biometric measurement using a biometric measurement device;
determining, via a processor, whether the biometric measurement corresponds to a previously acquired biometric measurement stored in a memory device; and
preventing, via the processor, the pulse oximetry sensor from obtaining physiological data when the biometric measurement does not correspond to the previously acquired biometric measurement.

18. The method of claim 17, comprising:
enabling, via the processor, the pulse oximetry sensor to obtain physiological data when the biometric measurement corresponds to the previously acquired biometric measurement;
associating, via the processor, the biometric data with the physiological data using the processor; and
encrypting, via the processor, the physiological data based on the biometric measurement using the processor.

19. The method of claim 17, comprising prompting, via the processor, acquisition of the biometric measurement via the biometric measurement device upon connection of the pulse oximetry sensor to a pulse oximetry monitor or upon powering on the pulse oximetry sensor.

20. The method of claim 17, comprising storing on the memory device the previously acquired biometric measurement of only a single patient.
